(19) United States
(12) Patent Application Publication
(10) Pub. No.: US 2026/0199473 A1
(43) Pub. Date: Jul. 16, 2026

(54) HOMEOPATHIC COMPOSITION FOR INCREASING THE VITAL ENERGY OF HUMAN BEINGS AND RESPECTIVE DOSAGE

(71) Applicant: Huang Wei LING, Franca (BR)

(72) Inventor: Huang Wei LING, Franca (BR)

(21) Appl. No.: 19/138,554

(22) PCT Filed: Aug. 2, 2024
(86) PCT No.: PCT/BR2024/000007

§ 371 Date: Jun. 12, 2025

(30) Foreign Application Priority Data
Oct. 10, 2023 (BR) BR102023021063-5

(51) Int. Cl. A61K 41/00

(52) U.S. Cl. CPC A61K 41/0004

(57) ABSTRACT

Refers to an homeopathic composition developed to treat various diseases that affect human beings such as emotional disorders, anxiety, depression, panic syndrome, insomnia, schizophrenia, autism, etc., in addition to all physical causes such as high blood pressure, myocardial infarction with or without obstruction, all autoimmune diseases such as rheumatoid arthritis, autoimmune hepatitis, thyroiditis, type 1 and 2 diabetes, all types of cancer, stroke and all types of community and hospital infections, consisting for a formulation of 1,000 ml, by 4.95 ML of Sulfur (sulfur); 4.95 ml of Calcium Carbonate (carbonic limestone); 4.95 ml of Silicon Oxide (silicea); 4.95 ml of Phosphorus (phosphorus); 4.95 ml of Sodium Chloride and 975.25 ml of 10% Apple Cider Vinegar are used as a dilution vehicle.

### INTRODUCTION

This descriptive report of the Invention Patent refers to the Homeopathic Composition for Increasing the Vital Energy of Human Beings and Respective Dosage, hereinafter referred to as Homeopathic Composition, which was developed to treat various diseases that affect human beings such as emotional disorders, anxiety, depression, panic syndrome, insomnia, schizophrenia, autism, etc., in addition to all physical causes such as high blood pressure, myocardial infarction with or without obstruction, all autoimmune diseases such as rheumatoid arthritis, autoimmune hepatitis, thyroiditis, type 1 and 2 diabetes, all types of cancer, stroke and all types of community and hospital infections.

In this way and to meet the intended objectives, the Homeopathic Composition presented here is formed by a composition formulated through sulfur (sulphur), calcium carbonate (calcarea carbonica), silicon oxide (silicea), phosphorus (phosphorus) and sodium chloride (natrum muriaticum), diluted in 10% apple cider vinegar.

### FIELD OF APPLICATION

The field of application of this Homeopathic Composition applies to various clinical situations of illness such as chronic non-communicable diseases, respiratory and allergic diseases, psychosomatic disorders, among others.

### PURPOSES

Among the main purposes of this Homeopathic Composition, we highlight the contribution to improving the quality of life of users, offering solutions to prevent, alleviate and cure acute and chronic diseases, in addition to reducing and/or eliminating the demand for hospital and emergency interventions.

### DESCRIPTION OF THE RELATED ART

Using a metaphor of a tree, it can be explained that it is composed of and divided into two parts, the part above ground level and the part below ground level, which is normally not visible.

The part above the ground is divided by the trunk and coming out of the trunk we see several branches, each branch representing a medical specialty and coming out of each branch we see several leaves, which represent each symptom or disease treated by each specialty.

Medicine practiced today treats the symptoms of all medical specialties at the leaf level, just as homeopathy is treating at the leaf level because it is treating the patient's symptoms and makes a diagnostic correlation according to the principles of occidental medicine.

In traditional Chinese medicine, the cause of the symptoms in each leaf in each specialty is not at the leaf level, but at the root level of the tree. This part is not understood by occidental medicine or homeopathy, as it was not taught in medical schools since 1910, the year in which changes were made to the curricula of medical schools in the United States and Canada, stating that anything that could be proven at the level of radiological and/or laboratory exams would be considered scientific.

In this way, there was a division of the human being into a materialized part and an energetic part (since everything that exists in the universe is composed of energy). It is worth noting that medical schools do not study this second part, as they do not even know that there is energy within the body, since they have always only treated the symptoms, but have never studied where diseases come from, resulting from energy imbalances present at the root level of the tree.

In Chinese medicine, all physical or emotional symptoms are caused by deficiencies or excesses in the root of the tree and can also be influenced by external pathogenic factors that both parties do not take into account in occidental medicine and, therefore, can often make erroneous diagnoses by not taking these factors into account.

At the root of the tree, there are two theories of traditional Chinese medicine that are important for treating most diseases, which are the theory of Yin and Yang and the theory of the five elements (Wood, Fire, Earth, Metal, Water). In this last theory, there are five elements in the universe that must be in harmony for balance to exist and within our body, the five elements are represented by our internal solid organs which are the LIVER, HEART, SPLEEN, LUNG and KIDNEY respectively.

Each internal solid organ is responsible for an external sensory organ, for example, the LIVER is responsible for the eye and vision. The HEART is responsible for our tongue and communication. The earth is responsible for the mouth and the sensation of taste of food. The LUNG is responsible for the nose and the sensation of smell of things. The KIDNEY is responsible for the ear and hearing.

Therefore, in pathologies of any alteration in these functions, treatment is given to the affected internal organs and not to the external organ, as is the case today.

The need to use homeopathy today, and to treat the root of all problems and not just the symptoms, is due to the low energy that almost the majority of patients present today, due to electromagnetic contamination caused by the modernization of the means of communication, leading to the reduction of our vital energy, leaving the majority of our patients immune deficient.

This general state of immunosuppression is also occurring in babies as soon as they are born and therefore, the use of these medications to replenish the energy of these organs is necessary in the general population today, as our lines of defense are compromised, leading to this global pandemic by SARS-CoV-2. The problem in this case is not the virus itself, but rather the normal lines of defense of the human being are compromised by this lack of general energy because if they were normal, the contact of the human being with the virus would not cause a problem because the defenses would be normal in our own body and could take charge of overcoming these external agents if our internal energy responsible for our defenses were normal, but today they are not.

According to traditional Chinese medicine, all internal massive organs are interconnected by an energy flow and one depends on the energy of the other. In the study, all internal organs are without energy, and it is necessary to charge the energy of these organs using highly diluted medicines, which in this case correspond to the homeopathic remedies Phosphorus, Sulphur, Calcarea carbonica, Silicea, Natrum muriaticum.

The LIVER receives energy from the Kidney; the KIDNEY receives energy from the LUNG; the LUNG receives energy from the SPLEEN, the SPLEEN receives energy from the HEART and finally the HEART receives energy from the LIVER, closing the cycle.

### REFERENCE CITED

It is worth noting that the Patent Application, Homeopathic Composition, has its own development characteristics. Prior art searches were conducted and the following documents were found:
Patent BR 10 2016 00518 4, deals with a resultant preparation of the method, composed of the inexhaustible source of energy from the universe. This method is a subtle energization process, it is a concentrated compound that benefits the necessary aspects of the individual, so there is no risk in using it.
Patent CO 6341564 filed on Aug. 29, 2008 refers to methods of topical application of an aqueous homeopathic composition for the treatment of various diseases and injuries, including deep tissue wounds and surgical wounds. The aqueous homeopathic composition is formulated with a high potency of at least 4000, maintained in an aqueous medium and applied topically to a body surface for an extended period of time to achieve therapeutic treatment.

### TECHNOLOGICAL ADVANCEMENT

Homeopathic Composition refers to the use of homeopathy to treat the root of all problems and not just the symptoms, increasing the energy that almost most patients have today, due to electromagnetic contamination due to the modernization of the means of communication, leading to the reduction of our vital energy, leaving most of our patients immunodeficient, and in this way, the Technological Advancement presented aims to promote the treatment of each organ with a specific homeopathy.

Furthermore, the use of the Homeopathic Composition now on the screen includes treatment that is linked to the root of all human problems, whether emotional or physical, since all problems in all specialties converge at the root of the human being, allowing the treatment of all emotional illnesses such as anxiety, depression, panic syndrome, insomnia, schizophrenia, autism, etc., in addition to all physical causes such as high blood pressure, myocardial infarction with or without obstruction, all autoimmune diseases such as rheumatoid arthritis, autoimmune hepatitis, thyroiditis, type 1 and 2 diabetes, all types of cancer, stroke, in addition to all types of community and hospital infections.

### DESCRIPTION OF THE INVENTION

The Homeopathic Composition presented herein was developed to treat various diseases that affect humans, such as emotional disorders, anxiety, depression, panic syndrome, insomnia, schizophrenia, autism, etc., in addition to all physical causes such as high blood pressure, myocardial infarction with or without obstruction, all autoimmune diseases, rheumatoid arthritis, autoimmune hepatitis, thyroiditis, type 1 and 2 diabetes, all types of cancer, stroke, as well as all types of community and hospital infections.

In accordance with its purposes, the Homeopathic Composition presented herein is formed by a composition formulated using sulfur (sulphur), calcium carbonate (calcarea carbonica), silicon oxide (silicea), phosphorus (phosphorus) and sodium chloride (natrum muriaticum), in the proportions described below.

### BEST WAY TO PREPARE

The preparation of the Homeopathic Composition involves the selection of the components, highlighting that for the formulation of 1,000 ml, 4.95 ML of Sulfur (sulfur); 4.95 ml of Calcium Carbonate (carbonic limestone); 4.95 ml of Silicon Oxide (silicea); 4.95 ml of Phosphorus (phosphorus); 4.95 ml of Sodium Chloride and 975.25 ml of 10% Apple Cider Vinegar are used as a dilution vehicle.

Regarding the preparation, we start by adding sulfur (Sulphur) to the dilution vehicle, secondly Calcarea carbonica, thirdly Silicea, fourthly Sodium Chloride and lastly we add Phosphorus. It is worth noting that as each component is added to the dilution vehicle, the partial combination is shaken for perfect dilution, then the next component is added and so on, with the entire process being carried out in the 30 ml amber glass bottle.

As stated above, all the components of the Homeopathic Composition are dissolved in a dilution vehicle, preferably 10% apple cider vinegar.

### PRESENTATION

The Homeopathic Composition is presented in the form of a solution.

### INITIAL STUDIES

When studies were carried out in the area of homeopathy, it became clear that the symptoms that homeopathic medicines treat are “memorized” by doctors, as there is no logical reasoning (beyond the simillimum theory) to choose one medicine or another (the mechanisms of action of homeopathy are still not understood). Today, there is an extensive arsenal of homeopathic medicines (more than 2,000) that need to be practically memorized by prescribing doctors, in order to know which medicine to use in each case presented by the patient.

The homeopathic formulation presented here is based on clinical reasoning and a patient was treated in 2006. The results obtained in this patient made it possible to establish a new way of treating patients, which involves treating energy imbalances (invisible to the naked eye) and not just the symptoms.

0 The 70-year-old black patient had a history of leg pain and was treated for 6 months with anti-inflammatories, but showed no improvement. Based on the analysis of his medical history and the treatment applied, instead of treating only his symptoms, the energy imbalance that was causing the symptoms was treated. He had a deficiency in Kidney Yang energy, as he felt cold in his extremities and had a blood deficiency because he did not have a daily bowel habit.

Ten acupuncture sessions were performed, and the patient returned for a reevaluation and reported that the pain had improved, but that he had also improved another condition that he had not told me about. This patient had been treated for glaucoma for the past 40 years, with no improvement in intraocular pressure, only by using eye drops to lower the pressure in his eye. For the first time in his life, his intraocular pressure reduced from 40 to 17 mmHg when his energy imbalances were treated, not just the symptoms.

### DOSAGE

A dosage of 5 drops of the composition diluted in water per day is applied to adults, starting at 30 CH. This initial potency is used to ensure that the weakened organism receives the initial dose of energy, thus scaling up to higher potencies This potency is normally used for 1 day or until the bottle of homeopathy is finished, always depending on the needs of each patient. If the patient needs higher potencies quickly, this potency can be administered for a shorter period and the next potency of 200 CH can be administered, which is administered for a longer period if the patient has physical problems such as gastritis, reflux, organ deficiencies, etc. A daily dose of this potency of 5 drops per day is normally maintained, however, if the patient still complains of weakness and indisposition, then the higher potency of 1000 CH is administered, giving a daily dose of 5 drops per day.

The higher potencies of 10,000 CH and 50,000 CH are used in more specific situations, such as menstrual uterine bleeding where the patient does not stop the menstrual flow. This situation is caused by a lack of energy and the formation of internal heat, which are stopped with the use of higher homeopathic potencies of this compound. In this case, the patient can use these higher potencies every 4 weeks, 20 drops in 20 ml of water. This procedure requires the previous scheme to be carried out beforehand, as a patient who is very energetically weak cannot receive a lot of energy at once and therefore must be done gradually, starting with the lower potencies until reaching the higher potencies. 032 Based on the above, we can see that Homeopathic Composition brings enormous advantages, as the choice of substances was made based on the concept of organotropism, thus applied: Phosphorus acting on the LIVER, Sulphur acting on the HEART, Calcárea carbonica acting on the SPLEEN, Silicea acting on the LUNG and Natrum muriaticum acting on the Kidney.

Based on the above, we can see that Homeopathic Composition brings enormous advantages, since the choice of substances was made based on the concept of organotropism, thus applied: Phosphorus acting on the LIVER, Sulphur acting on the HEART, Calcárea carbonica acting on the SPLEEN, Silicea acting on the LUNG and Natrum muriaticum acting on the Kidney.

Therefore, the administration protocol is often short, when administered only once a month, and being easier, more effective and with fewer side effects, leaving the protocol only at 30 CH, 200 CH, 1000 CH and leaving the potencies of 10,000 CH and 50,000 CH for specific cases, in which patients, when they reach 1000 CH, still complain of not having improved yet.

### CONCLUSION

It can be seen from everything that has been described that, at a biophysical level, the action of these dynamized substances, following criteria correlated to the concepts of Flow of Vital Energy and Five Elements, give the works a character of innovation and originality, since we also do not find in the literature dedicated to Traditional Chinese Medicine the mention of the application of dynamized inputs as a therapeutic strategy, which is why the Homeopathic Composition for Increasing the Vital Energy of the Human Being and Respective Dosage fits perfectly within the rules governing the Invention Patent.

1. A “HOMEOPATHIC COMPOSITION FOR INCREASING THE VITAL ENERGY OF HUMAN BEINGS AND RESPECTIVE DOSAGE”, developed to treat various diseases that affect human beings such as emotional disorders, anxiety, depression, panic syndrome, insomnia, schizophrenia, autism, etc., in addition to all physical causes such as high blood pressure, myocardial infarction with or without obstruction, all autoimmune diseases such as rheumatoid arthritis, autoimmune hepatitis, thyroiditis, type 1 and 2 diabetes, all types of cancer, stroke and all types of community and hospital infections, being characterized by consisting of, for a formulation of 1,000 ml, by 4.95 ML of Sulfur (sulfur); 4.95 ml of Calcium Carbonate (carbonic limestone); 4.95 ml of Silicon Oxide (silicea); 4.95 ml of Phosphorus (phosphorus); 4.95 ml of Sodium Chloride and 975.25 ml of 10% Apple Cider Vinegar are used as a dilution vehicle.

2. The “HOMEOPATHIC COMPOSITION FOR INCREASING THE VITAL ENERGY OF HUMAN BEINGS AND RESPECTIVE DOSAGE”, according to claim 1, characterized by the order of dilution, first adding sulfur (Sulphur) to the dilution vehicle, secondly Calcarea carbonica, thirdly Silicea, fourthly Sodium Chloride and lastly we add Phosphorus.

3. The “HOMEOPATHIC COMPOSITION FOR INCREASING THE VITAL ENERGY OF HUMAN BEINGS AND RESPECTIVE DOSAGE”, according to claim 1, characterized by each component is added to the dilution vehicle, the partial combination is shaken for perfect dilution, then the next component is added and so on, with the entire process being carried out in the 30 ml amber glass bottle.
